(19) United States
(12) Patent Application Publication
(10) Pub. No.: US 2012/0294947 A1
(43) Pub. Date: Nov. 22, 2012

(54) Oral Preparation Having Improved Quality

(75) Inventors: Kennichiro Kuninobu, Osaka-shi (JP); Yohei Hoashi, Osaka-shi (JP); Naohisa Katayama, Osaka-shi (JP); Toshiya Kai, Osaka-shi (JP)

(73) Assignee: NIPRO CORPORATION, Osaka-shi, Osaka (JP)

(21) Appl. No.: 13/519,193

(22) PCT Filed: Dec. 28, 2010
(86) PCT No.: PCT/JP2010/073763

§ 371 Date: Jun. 26, 2012

(30) Foreign Application Priority Data
Dec. 28, 2009 (JP) 2009-298548

(51) Int. Cl. A61K 9/14; A61K 31/437; A61J 3/10; A61K 31/4422; A61P 25/28; A61K 31/445; A61K 31/519

(52) U.S. Cl. 424/497; 424/490; 514/319; 514/300; 514/259.41; 514/356; 264/109

(57) ABSTRACT

Provided is an oral preparation that contains a medicinal component having an unpleasant taste and that has better qualities, for example, generation of an analogue can be reduced, better than oral preparations that are produced by conventional techniques and that contain a medicinal component having an unpleasant taste. This objective is achieved by adding a coating agent on the medicinal component and a disintegrator that has a carboxymethyl group.

### TECHNICAL FIELD

The present invention relates to an oral preparation with which generation of an analogue resulting from a medicinal component is suppressed, the unpleasant taste such as bitterness resulting from the medicinal component is reduced, the dissolution behavior of the medicinal component is controlled, and which has reduced friability, and the present invention also relates to a production method therefor.

### BACKGROUND ART

Some medicinal components contained in oral preparations are problematic in that they are partially lost as time passes due to the decomposition reaction or the like caused by, for example, light. Also, some medicinal components contained in oral preparations are problematic in that the recipient suffers when taking a preparation because some medicinal components have a bitter taste. Furthermore, some oral preparations and, in particular, solid preparations are problematic in that they have a high level of friability that allows, for example, a part of solid preparations to be chipped off by, for example, an external impact received after being shaped. Accordingly, it is difficult to use an automatic packaging machine at the drug dispensing site, resulting in problems such as significantly poor drug dispensing efficiency.

One example of medicinal components that have such problems is donepezil. Donepezil is a therapeutic agent for Alzheimer-type dementia and has a structural formula as shown in Formula I below.

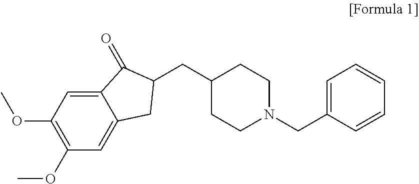

A tablet that contains donepezil hydrochloride as a medicinal component (hereinafter referred to as a donepezil hydrochloride-containing tablet) is already disclosed in Non-Patent Document 1. However, the donepezil hydrochloride-containing tablet described in Non-Patent Document 1 is problematic in that, in the case where it is stored for a long period of time, donepezil hydrochloride, which is a medicinal component unstable to light, is lost. Also, the donepezil hydrochloride-containing tablet described in Non-Patent Document 1 has a problem of a high level of friability.

Also, Patent Document 1 discloses an oral pharmaceutical composition containing an anionic high-molecular substance and a basic medicinal substance that has an unpleasant taste. Donepezil hydrochloride is described as one of the basic medicinal substances that have an unpleasant taste. Patent Document 1 describes, as the effect of the invention, that the unpleasant taste such as bitterness and numbness brought about by donepezil hydrochloride can be masked.

However, regarding the oral donepezil hydrochloride pharmaceutical composition disclosed in Patent Document 1, Patent Document 1 is silent as to the effect of the invention other than the unpleasant-taste masking effect.

### PRIOR ART DOCUMENTS

### Patent Documents

Patent Document 1: Japanese Laid-Open Patent Publication No. H11-228450

### Non-Patent Documents

Non-Patent Document 1: Package insert of Aricept (registered trademark) D tablets

### SUMMARY OF INVENTION

### Problems to be Solved by the Invention

An object of the present invention is to provide a donepezil hydrochloride-containing oral preparation having better qualities than donepezil hydrochloride-containing tablets produced by conventional techniques.

Specifically, for example, when the donepezil hydrochloride-containing tablet described in Non-Patent Document 1 is photo-irradiated at 300000 lux·hr, a donepezil analogue in the tablet is increased to about 0.73% by weight, and accordingly an object is, for example, to make it possible to create a method for producing a novel donepezil hydrochloride-containing oral preparation with which this increase of the donepezil analogue can be suppressed at low cost, and in a simple manner and thus to provide a donepezil hydrochloride-containing oral preparation in which the donepezil hydrochloride content is not reduced over a longer period of time and which is safe for patients.

### Means for Solving the Problems

The summary of the present invention is as follows.

(1) An oral preparation comprising a medicinal substance having an unpleasant taste, wherein the preparation is obtained through steps 1 to 6 below:

1. a step of obtaining a mixture containing a medicinal substance having an unpleasant taste and a first additive,

2. a step of dissolving or suspending a binder in a granulation liquid preparation solvent to obtain a granulation liquid,

3. a step of adding the granulation liquid to the mixture and carrying out granulation with an agitation granulator to obtain granules,

4. a step of dissolving or suspending a coating agent, a lubricant, and/or a low-viscosity binder in a spray liquid preparation solvent to obtain a spray liquid,

5. a step of spraying the spray liquid onto the granules to coat the granules to obtain coated granules, and

6. a step of mixing the coated granules with a second additive and a disintegrator that has a carboxymethyl group, and tableting the mixture.

(2) The oral preparation according to (1), wherein the medicinal substance having an unpleasant taste contains one medicinal substance selected from donepezil hydrochloride, zolpidem tartrate, risperidone, or amlodipine besilate.

(3) The oral preparation according to (1), wherein the medicinal substance having an unpleasant taste is donepezil hydrochloride.

(4) The oral preparation according to (1), wherein the first additive is an excipient.

(5) The oral preparation according to (1), wherein the coating agent is an acrylic polymer-based coating agent.

(6) The oral preparation according to (1), wherein the coating agent is Eudragit NE.

(7) The oral preparation according to (1), wherein the disintegrator that has a carboxymethyl group is carmellose, carmellose sodium, carmellose calcium, croscarmellose sodium, sodium carboxymethyl starch, or carboxy methyl ethyl cellulose.

(8) A method for producing an oral preparation containing a medicinal substance having an unpleasant taste, which comprises steps 1 to 6 below:

1. a step of obtaining a mixture containing a medicinal substance having an unpleasant taste and a first additive,

2. a step of dissolving or suspending a binder in a granulation liquid preparation solvent to obtain a granulation liquid,

3. a step of adding the granulation liquid to the mixture and carrying out granulation with an agitation granulator to obtain granules,

4. a step of dissolving or suspending a coating agent, a lubricant, and/or a low-viscosity binder in a spray liquid preparation solvent to obtain a spray liquid,

5. a step of spraying the spray liquid onto the granules to coat the granules to obtain coated granules, and

6. a step of mixing the coated granules with a second additive and a disintegrator that has a carboxymethyl group, and tableting the mixture.

The present invention will now be described in detail below.

Donepezil herein refers to the compound represented by Formula I above. Donepezil suppresses decomposition of acetylcholine in the brain by inhibiting acetylcholinesterase. Due to this effect, donepezil exhibits a pharmacological effect of preventing a decrease of the activity of the cholinergic nervous system in the brain, and has been used as a therapeutic agent of Alzheimer-type dementia.

Examples of the medicinal substance that has an unpleasant taste usable in the oral preparation of the present invention include medicinal substances that exhibit a strong bitter taste when administered, which makes it difficult to take them.

Specific examples include donepezil hydrochloride, zolpidem tartrate, risperidone, amlodipine besilate, and the like.

The medicinal substance most preferable in the present invention that has an unpleasant taste is donepezil hydrochloride.

Examples of the first additive usable in the oral preparation of the present invention include diluting agents, binders, lubricants, disintegrators, plasticizers, antioxidants, antistatic agents, pH adjustors, fluidizers, surfactants, coloring agents, and the like.

Examples of the second additive usable in the oral preparation of the present invention include diluting agents, lubricants, disintegrators, antioxidants, antistatic agents, pH adjustors, fluidizers, surfactants, coloring agents, and the like.

Examples of diluting agents usable in the oral preparation of the present invention include sugar alcohols, saccharides, starches or derivatives thereof, and the like.

Specific examples of sugar alcohols usable in the oral preparation of the present invention include mannitol, erythritol, xylitol, maltitol, sorbitol, and the like. More preferable are mannitol and xylitol, and most preferable is mannitol.

Specific examples of saccharides usable in the oral preparation of the present invention include hydrates, non-hydrates, or the like of lactose, sucrose, saccharose, trehalose, fructose, glucose, and the like. More preferable are lactose and sucrose, and most preferable is lactose.

Specific examples of starches or derivatives thereof usable in the oral preparation of the present invention include corn starch, potato starch, and the like.

Specific examples of binders usable in the oral preparation of the present invention include sodium alginate, ethylcellulose, carrageenan, gelatin, hydroxypropylcellulose, polyvinylpyrrolidone, carboxy vinyl polymer, agar, copolyvidone, purified shellac, dextrin, hydroxyethyl cellulose, hydroxyethyl methyl cellulose, hydroxypropyl starch, hydroxypropyl cellulose, vinylpyrrolidone-vinyl acetate copolymer, hypromellose, partially pregelatinized starch, pullulan, pectin, polyvinyl alcohol-polyethylene glycol graft copolymer, povidone, polyvinyl alcohol, methacrylic acid copolymer L, methacrylic acid copolymer LD, methacrylic acid copolymer S, methylcellulose, and the like.

Examples of binders preferably used during the preparation of the granulation liquid of the present invention include hydroxypropyl cellulose, polyvinylpyrrolidone, hypromellose, and polyvinyl alcohol

Examples of lubricants used in the present invention include talc, titanium oxide, glycerine, glycerol fatty acid ester, wheat starch, sucrose fatty acid ester, stearyl alcohol, stearic acid and salts thereof, cetanol, gelatin, polyoxyethylene-polyoxypropylene glycols, polysorbates, macrogols, glyceryl monostearate, sodium lauryl sulfate, and the like.

Examples of lubricants preferably used during the preparation of the spray liquid of the present invention include magnesium stearate, talc, and titanium oxide.

Examples of disintegrators used in the present invention include corn starch, starch, crystalline cellulose, stearic acid and salts thereof, talc, crospovidone, cellulose or derivatives thereof, and the like.

Examples of disintegrator preferably used in the present invention include crystalline cellulose and corn starch.

Examples of plasticizers used in the present invention include triacetin, triethyl citrate, polypropylene glycol, polyethylene glycol, glycerine, polysorbate 80, diethyl sebacate, dibutyl sebacate, stearic acid, and the like.

Examples of antioxidants used in the present invention include tocopherol, ascorbic acid, sodium hydrogen sulfite, sodium sulfite, sodium edetate, erythorbic acid, cysteine hydrochloride, dried sodium sulfite, citric acid hydrate, dibutylhydroxytoluene, soybean lecithin, natural vitamin E, tocopherol, sodium pyrosulfite, butylhydroxyanisol, propyl gallate, and the like.

Examples of antistatic agents used in the present invention include hydrous silicon oxide, light anhydrous silicon, talc, and the like.

Examples of pH adjusters used in the present invention include citric acid and salts thereof, phosphoric acid and salts thereof, carbonic acid and salts thereof, tartaric acid and salts thereof, fumaric acid and salts thereof, acetic acid and salts thereof, amino acids and salts thereof, succinic acid and salts thereof, lactic acid and salts thereof, and the like.

Examples of fluidizers used in the present invention include light anhydrous silicic acid, hydrous silicon oxide, titanium oxide, talc, stearic acid and salts thereof, heavy silicic acid anhydride, and the like.

Examples of fluidizers preferably used in the present invention include light anhydrous silicic acid, titanium oxide, talc, and the like.

Examples of surfactants used in the present invention include phospholipid, glycerol fatty acid ester, polyoxyethylene fatty acid ester, sorbitan fatty acid ester, polyethylene glycol fatty acid ester, polyoxyethylene hydrogenated castor oil, polyoxyethylene alkyl ether, sucrose fatty acid ester, sodium lauryl sulfate, polysorbates, sodium hydrogen phosphates, potassium hydrogen phosphates, and the like.

Examples of coloring agents used in the present invention include iron sesquioxide, yellow iron sesquioxide, tar-based color, aluminum chelate, titanium oxide, talc, and the like.

Examples of coloring agents preferably used in the present invention include titanium oxide, talc, iron sesquioxide, tar-based color, and the like.

Examples of solvents for the preparation of the granulation liquid usable in the oral preparation of the present invention include water, ethanol, isopropyl alcohol, and the like.

Specific examples of coating agents usable in the oral preparation of the present invention include ethyl acrylate-methyl methacrylate copolymer, aminoalkyl methacrylate copolymer, ethyl cellulose, carboxyvinyl polymer, methacrylic acid copolymer, dimethylaminoethyl methacrylate-methyl methacrylate copolymer, and the like.

Examples of coating agents preferably used in the present invention include acrylic polymer-based coating agents such as Eudragit NE30D and Eudragit L.

Specific examples of low-viscosity binders usable in the oral preparation of the present invention include sodium alginate, ethyl cellulose, carrageenan, gelatin, hydroxypropyl cellulose, polyvinylpyrrolidone, carboxyvinyl polymer, agar, copolyvidone, purified shellac, dextrin, hydroxyethyl cellulose, hydroxyethyl methyl cellulose, hydroxypropyl starch, hydroxypropyl cellulose, vinylpyrrolidone-vinyl acetate copolymer, hypromellose, partially pregelatinized starch, pullulan, pectin, polyvinyl alcohol-polyethylene glycol graft copolymer, povidone, polyvinyl alcohol, methacrylic acid copolymer L, methacrylic acid copolymer LD, methacrylic acid copolymer S, methylcellulose, and the like.

Examples of low-viscosity binders preferably used in the present invention include methylcellulose, hypromellose, and the like.

Specific examples of disintegrators that have a carboxymethyl group usable in the oral preparation of the present invention include carmellose, carmellose sodium, carmellose calcium, croscarmellose sodium, sodium carboxymethyl starch, carboxy methyl ethyl cellulose, and the like.

Examples of disintegrators that have a carboxymethyl group preferably used in the present invention include carmellose, croscarmellose sodium, carmellose calcium, and sodium carboxymethyl starch.

Examples of solvents for the preparation of the spray liquid usable in the oral preparation of the present invention include water, ethanol, and the like.

Specific examples of the oral preparation in the present invention include tablets, powders, capsules, granules, and the like. The oral preparation most preferably used in the present invention is a tablet.

It is possible to carry out agitation and granulation in the present invention with a generally known agitation granulator. Examples of generally known agitation granulators include vertical granulators, high-speed mixers, and the like.

The tableting method for tablets to be provided by the present invention is not particularly limited as long as the effect of the present invention is demonstrated. Examples of the tableting method in the present invention include dry indirect compression method, wet indirect compression method, dry direct compression method, and the like.

Tablets provided by the present invention are shaped using, for example, a single-punch tableting machine, a rotary tableting press machine, or the like. The pressure for tableting is usually 4 to 20 kN/cm². The shape of the solid preparation of the present invention is not particularly limited, and specific examples include shapes such as round, caplet, doughnut and oblong as well as multilayered tablets and cored tablets. Moreover, the tablets may be provided, if necessary, with distinguishing characters, symbols, or marks and, moreover, may be provided with a line along which tablets can be broken.

The donepezil analogue, which can be reduced by the present invention, can be quantified as follows.

From a chromatogram obtained by HPLC, the sum of the peak areas excluding the main-peak area and the additive-derived peak areas is calculated, the resulting value is divided by the value of the main-peak, the obtained value is regarded as the amount of the analogue, and comparison/evaluation is carried out. More specifically, comparison/evaluation can be carried out using the test method described in paragraph [0055].

### Effects of Invention

The present invention has made it possible to obtain a donepezil hydrochloride-containing tablet with which generation of a donepezil analogue in the tablet, which occurs through photo-irradiation for a long period of time, can be less than with conventional donepezil hydrochloride-containing tablets.

Also, the present invention has made it possible to reduce an unpleasant taste such as bitterness resulting from donepezil hydrochloride felt after taking a donepezil hydrochloride-containing tablet in a sensory test.

Also, the present invention has made it possible to obtain a donepezil hydrochloride-containing tablet with which the dissolution behavior of donepezil hydrochloride immediately after taking the donepezil hydrochloride-containing tablet can be more controlled compared with conventional donepezil hydrochloride-containing tablets.

Also, the present invention has made it possible to obtain a donepezil hydrochloride-containing tablet having a lower level of friability than conventional donepezil hydrochloride-containing tablets.

Also, the present invention has made it possible to obtain a donepezil hydrochloride-containing tablet having a lower level of porosity.

### MODE FOR CARRYING OUT THE INVENTION

The best mode for carrying out the invention will now be disclosed below.

### EXAMPLES

### Example 1

After 65 parts by weight of donepezil hydrochloride and 110 parts by weight of D-mannitol were mixed to give a mixture, the mixture was introduced into an agitation granulator (VG-05, manufactured by Powrex Corporation), and a granulation liquid composed of 2 parts by weight of hypromellose and 20 parts by weight of purified water was added, to give granules. The granules were dried in a fluidized-bed drier (Multiplex MP-01, manufactured by Powrex Corporation), and pulverization and particle size regulation were carried out with a power mill. The dried granules were sprayed with a spray liquid composed of 150 parts by weight of Eudragit NE30D, 25 parts by weight of talc, 25 parts by weight of titanium oxide, 10 parts by weight of methylcellulose, and 200 parts by weight of purified water to give coated granules. Two parts by weight of light anhydrous silicic acid, 10 parts by weight of carmellose, 2 parts by weight of magnesium stearate, 10 parts by weight of corn starch, 5 parts by weight of crystalline cellulose, and 101 parts by weight of D-mannitol were added to and mixed with 20 parts by weight of the prepared coated granules, and tableting was carried out with a rotary tableting machine (VIRGO, manufactured by Kikusui Seisakusho Ltd.).

### Example 2

Two parts by weight of light anhydrous silicic acid, 10 parts by weight of carmellose calcium, 2 parts by weight of magnesium stearate, 10 parts by weight of corn starch, 5 parts by weight of crystalline cellulose, and 101 parts by weight of D-mannitol were added to and mixed with 20 parts by weight of the coated granules as prepared in Example 1, and tableting was carried out with a rotary tableting machine (VIRGO, manufactured by Kikusui Seisakusho Ltd.).

### Example 3

Ten parts by weight of sodium carboxymethyl starch, 2 parts by weight of light anhydrous silicic acid, 2 parts by weight of magnesium stearate, 10 parts by weight of corn starch, 5 parts by weight of crystalline cellulose, and 101 parts by weight of D-mannitol were added to and mixed with 20 parts by weight of the coated granules as prepared in Example 1, and tableting was carried out with a rotary tableting machine (VIRGO, manufactured by Kikusui Seisakusho Ltd.).

### Example 4

After 65 parts by weight of donepezil hydrochloride and 110 parts by weight of D-mannitol were mixed to give a mixture, the mixture was introduced into an agitation granulator (high-speed mixer FS-02, manufactured by Fukae Seisakusho), and a granulation liquid composed of 2 parts by weight of hydroxypropyl cellulose and 20 parts by weight of purified water was added, to give granules. The granules were dried in a fluidized-bed drier (Multiplex MP-01, manufactured by Powrex Corporation), and pulverization and particle size regulation were carried out with a power mill. The dried granules were sprayed with a spray liquid composed of 150 parts by weight of Eudragit NE30D, 50 parts by weight of titanium oxide, 10 parts by weight of hypromellose, and 200 parts by weight of purified water to give coated granules. Two parts by weight of light anhydrous silicic acid, 10 parts by weight of carmellose, 2 parts by weight of magnesium stearate, 10 parts by weight of corn starch, 5 parts by weight of crystalline cellulose, and 101 parts by weight of D-mannitol were added to and mixed with 20 parts by weight of the prepared coated granules, and tableting was carried out with a rotary tableting machine (VIRGO, manufactured by Kikusui Seisakusho Ltd.).

### Example 5

After 50 parts by weight of zolpidem tartrate and 50 parts by weight of lactose hydrate were mixed to give a mixture, the mixture was introduced into an agitation granulator (VG-05, manufactured by Powrex Corporation), and a granulation liquid composed of 2 parts by weight of methylcellulose and 20 parts by weight of purified water was added, to give granules. The granules were dried in a fluidized-bed drier (Multiplex MP-01, manufactured by Powrex Corporation), and pulverization and particle size regulation were carried out with a power mill. The dried granules were sprayed with a spray liquid composed of 150 parts by weight of Eudragit L30D55, 50 parts by weight of titanium oxide, 5 parts by weight of triethyl citrate, and 200 parts by weight of purified water to give coated granules. Thirty parts by weight of croscarmellose sodium, 2 parts by weight of magnesium stearate, and 90 parts by weight of D-mannitol were added to and mixed with 30 parts by weight of the prepared coated granules, and tableting was carried out with a rotary tableting machine (VIRGO, manufactured by Kikusui Seisakusho Ltd.).

### Comparative Example 1

Ten parts by weight of crospovidone, 2 parts by weight of light anhydrous silicic acid, 2 parts by weight of magnesium stearate, 10 parts by weight of corn starch, 5 parts by weight of crystalline cellulose, and 101 parts by weight of D-mannitol were added to and mixed with 20 parts by weight of the coated granules as prepared in Example 1, and tableting was carried out with a rotary tableting machine (VIRGO, manufactured by Kikusui Seisakusho Ltd.).

### Comparative Example 2

Ten parts by weight of crystalline cellulose, 2 parts by weight of magnesium stearate, and 118 parts by weight of D-mannitol were added to and mixed with 20 parts by weight of the coated granules as prepared in Example 1, and tableting was carried out with a rotary tableting machine (VIRGO, manufactured by Kikusui Seisakusho Ltd.).

### Comparative Example 3

Two parts by weight of magnesium stearate, and 128 parts by weight of F-Melt Type C were added to and mixed with 20 parts by weight of the coated granules as prepared in Example 1, and tableting was carried out with a rotary tableting machine (VIRGO, manufactured by Kikusui Seisakusho Ltd.).

### Comparative Example 4

After 65 parts by weight of donepezil hydrochloride and 110 parts by weight of D-mannitol were mixed to give a mixture, the mixture was introduced into a fluidized-bed drier granulator (Multiplex MP-01, manufactured by Powrex Corporation), and a granulation liquid composed of 3 parts by weight of hydroxypropyl cellulose and 50 parts by weight of purified water was added, to give granules. Next, the granules were subjected to pulverization and particle size regulation in a power mill. The granules after pulverization and particle size regulation were sprayed with a spray liquid composed of 150 parts by weight of Eudragit NE30D, 50 parts by weight of talc, and 200 parts by weight of purified water to give coated granules. Two parts by weight of light anhydrous silicic acid, 10 parts by weight of carmellose, 2 parts by weight of magnesium stearate, 10 parts by weight of corn starch, 5 parts by weight of crystalline cellulose, and 101 parts by weight of D-mannitol were added to and mixed with 20 parts by weight of the prepared coated granules, and tableting was carried out with a rotary tableting machine (VIRGO, manufactured by Kikusui Seisakusho Ltd.).

### Comparative Example 5

Thirty parts by weight of carmellose sodium, 2 parts by weight of magnesium stearate, and 98 parts by weight of D-mannitol were added to and mixed with 20 parts by weight of the dried coated granules as prepared in Example 5, and tableting was carried out with a rotary tableting machine (VIRGO, manufactured by Kikusui Seisakusho Ltd.).

### Comparative Example 6

Thirty parts by weight of carmellose, 5 parts by weight of magnesium stearate, and 155 parts by weight of D-mannitol were added to and mixed with 30 parts by weight of the dried coated granules as prepared in Example 4, and tableting was carried out with a rotary tableting machine (VIRGO, manufactured by Kikusui Seisakusho Ltd.).

### Comparative Example 7

Aricept (registered trademark) 10 mg D tablets (produced and distributed by Eisai Co., Ltd.) were used.

### Test Example 1

### Test for Measurement of Analogue Content after Long-Term Storage Under Photo-Irradiated Conditions

The donepezil hydrochloride-containing oral preparations obtained in Examples 1 to 4 and Comparative Example 7 were each placed in a photostability test chamber (LTB-180C type, manufactured by Nagano Science Co., Ltd.), and irradiated at an illuminance of 1000 lux/hr under a D65 lamp so as to attain a cumulative illuminance of 300000 lux/hr. Thereafter, each sample was dissolved in a solution for use as a mobile phase such that the concentration of donepezil hydrochloride was 1 mg/mL. The used mobile phase was a solution prepared by mixing an aqueous 1-decanesulfonic acid solution and acetonitrile and perchloric acid in a ratio of 1300:700:1. The peak area ratio of analogue to donepezil hydrochloride was calculated using a test instrument (high-speed liquid chromatograph: LC-20A, manufactured by Shimadzu Corporation) and a DV detector as a detector while maintaining the temperature of a column (Mightsil ODS RP-18 GP 150-4.6), into which a sample was introduced, at 40° C. under conditions of the amount of sample introduced into the column of 10 μL, the flow rate of 1.3 mL/min, and the measurement wavelength of 271 nm to quantify the analogue. Test results are shown in Table 1.

TABLE 1

| | Analogue Content |
|---|---|
| | (% by weight) |
| Example 1 | 0.36 |
| Example 2 | 0.32 |
| Example 3 | 0.32 |
| Example 4 | 0.25 |
| Comparative | 0.73 |
| Example 7 | |

It is clear from these results that, with donepezil hydrochloride-containing tablets of the present invention, the analogue content can be lower than that with a conventional donepezil hydrochloride-containing tablet.

### Test Example 2

### Sensory Test of Oral Preparation

The taste of a tablet until it completely dissolved in saliva in the mouth of healthy male adults (27 years old, 175 cm height, 65 kg body weight) was evaluated. The test was carried out twice, and results of comprehensive evaluation of the test that was carried out twice are shown in Table 2.

TABLE 2

| | Evaluation of | Disintegration |
|---|---|---|
| | Bitterness | Time (sec.) |
| Example 1 | ± | 20 |
| Example 2 | − | 18 |
| Example 3 | − | 21 |
| Example 4 | ± | 23 |
| Example 5 | − | 24 |
| Comparative | + | 20 |
| Example 1 | | |
| Comparative | ++ | 25 |
| Example 2 | | |
| Comparative | + | 30 |
| Example 3 | | |
| Comparative | + | 29 |
| Example 4 | | |
| Comparative | ++ | 112 |
| Example 5 | | |
| Comparative | + | 30 |
| Example 6 | | |
| Comparative | + | 20 |
| Example 7 | | |

−: Absolutely no unpleasant taste or bitterness was felt.
±: Slight unpleasant taste or bitterness was felt.
+: Unpleasant taste or bitterness was felt.
++: Considerable unpleasant taste or bitterness was felt.

It is clear from these results that an unpleasant taste such as bitterness resulting from the medicinal component donepezil hydrochloride or zolpidem tartrate is reduced in oral preparations of the present invention.

### Test Example 3

### Dissolution Test of Oral Preparation

Three tablets prepared in each of Examples 1 to 3 and Comparative Examples 1 to 3 were subjected to a comparison of the dissolution rate (%) of the medicinal component by the upright-inverted syringe method. Values measured 30 seconds after the beginning of the test were regarded as dissolution rates and shown in Table 3.

The “upright-inverted syringe method” herein uses a simple dissolution test method (the upright-inverted syringe method) of Nakamura et al. that mimics the oral cavity. This method enables a measurement of the amount of a drug dissolved and an evaluation of bitter taste masking in comparison with a threshold of feeling a bitter taste (Reference: Yasuhiko Nakamura et at, “Ryuushi Sekkei To Seizai Gijyutsu (Particle Designing and Formulation Technique)” 121-128 (1993)). The upright-inverted syringe method herein is carried out as follows.

(Test on Donepezil Hydrochloride-Containing Particles)

Provide a 10 mL plastic syringe (manufactured by Nipro) to the end of which a 25 mm size filter having a pore size of 0.22 μm to 0.45 μm (Ekicrodisc manufactured by Pall Corporation) is attached, with the fluid outlet at the end of the filter being wrapped and blocked with a thermoplastic film (PARAFILM (registered trademark) M) to prevent fluid leakage. Introduce into this 10 mL glass syringe a tablet corresponding to 15 mg of donepezil hydrochloride and 10 mL of water maintained at 37±1° C. Attach a plunger at the same time, turn the syringe into an upright or inverted position at a rate of every 3 seconds for 30 seconds for a total of 10 times so that the PARAFILM detaches and filtration is carried out with the filter; discard the first 5 mL of the filtrate; recover the remaining filtrate; and measure the amount of donepezil hydrochloride.

Measurement of the amount of donepezil hydrochloride can be carried as follows.

The recovered filtrate is used as a test solution. Separately, about 150 mg of a donepezil hydrochloride reference standard that has been dried at 105° C. for 2 hours under reduced pressure is precisely weighed out, water as used for dissolution in this test is added, ultrasonic irradiation is carried out to dissolve the reference standard, and water is further added to attain 100 mL precisely, thus giving a standard solution. Using this standard solution and test solution and a UV-V is absorption spectrometer (U-3300, manufactured by Hitachi, Ltd.) as a detector, 4 mL of a sample is introduced into a 10 mm×10 mm quartz cell, and spectrophotometry is carried out at a measurement wavelength of 271 nm. Using the numerical value of the detected donepezil hydrochloride, the amount of dissolution by a simple dissolution test (the syringe upright-inverted method) is calculated by Equation (1) below. The obtained test results are shown in Table 3.

Rate (%) of dissolution by upright-inverted syringe method=Ws×At/As×⅔ (1)

where

Ws: Amount of donepezil hydrochloride reference standard (mg)

At: Absorbance of test solution

As: Absorbance of standard solution

TABLE 3

| | Dissolution Rate (%) |
|---|---|
| Example 1 | 22 |
| Example 2 | 7 |
| Example 3 | 16 |
| Comparative | 54 |
| Example 1 | |
| Comparative | 53 |
| Example 2 | |
| Comparative | 49 |
| Example 3 | |

It is clear from these results that with donepezil hydrochloride-containing tablets of the present invention, the dissolution behavior of donepezil hydrochloride immediately after administration can be controlled.

### Test Example 4

### Test for Measurement of Friability of Oral Preparations

The donepezil hydrochloride-containing oral preparations obtained in Examples 1, 4, and 5 and Comparative Example 7 were subjected to a test in accordance with a tablet friability testing method (The Japanese Pharmacopoeia Fifteenth Edition) to compare their level of friability (%). The results are shown in Table 4.

TABLE 4

| | Level of Friability |
|---|---|
| | (%) |
| Example 1 | 0.07 |
| Example 4 | 0.08 |
| Example 5 | 0.08 |
| Comparative | 0.34 |
| Example 7 | |

These results made it clear that oral preparations of the present invention have a lower level of friability than a conventional oral preparation.

### Test Example 5

### Test for Determination of Porosity of Oral Preparations

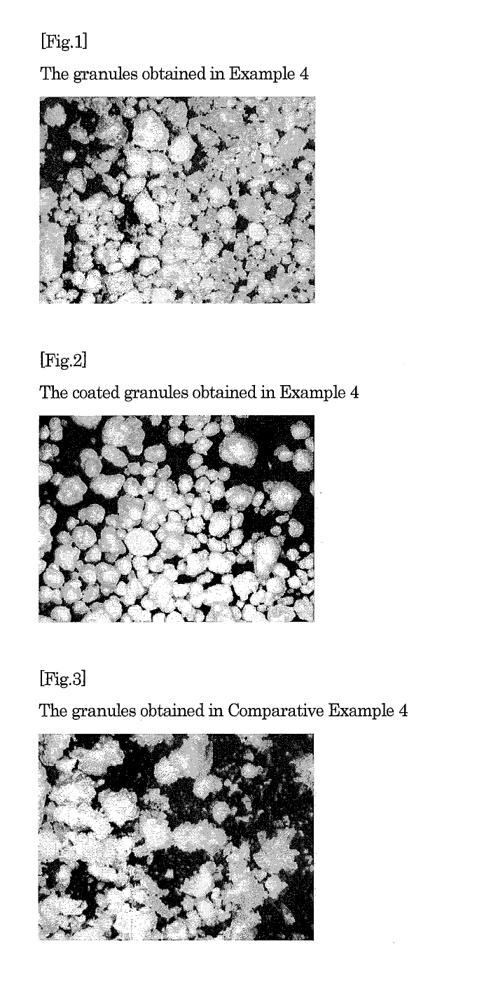

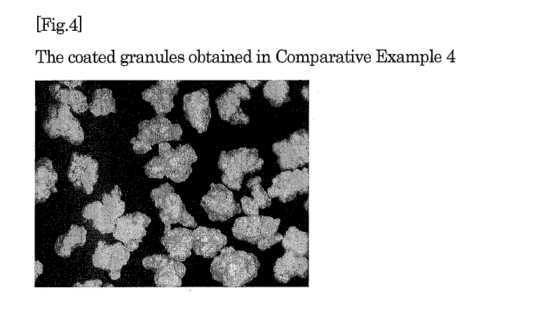

Enlarged images of granules and coated granules obtained in both Example 4 and Comparative Example 4 are presented in FIGS. 1 to 4 below.

These results made it clear that the donepezil hydrochloride-containing tablets of the present invention have reduced porosity.

### INDUSTRIAL APPLICABILITY

The present invention has made it possible to provide a novel oral preparation that has better qualities from a number of different perspectives compared with conventional oral preparations having an unpleasant taste. Accordingly, it has become possible to provide an oral preparation having an unpleasant taste that is safer to patients who receive the preparation.

1-8. (canceled)

9. An oral preparation comprising coated granules, a second additive and a disintegrator,
wherein the coated granules are granules coated with a coating agent and the coated granules contain a medicinal substance having an unpleasant taste, a first additive and a binder.

10. The oral preparation according to claim 9, wherein the medicinal substance having an unpleasant taste contains one medicinal substance selected from donepezil hydrochloride, zolpidem tartrate, risperidone, or amlodipine besilate.

11. The oral preparation according to claim 10, wherein the medicinal substance having an unpleasant taste is donepezil hydrochloride.

12. The oral preparation according to claim 9, wherein the first additive is an excipient.

13. The oral preparation according to claim 9, wherein the coated granules are spray-coated granules with a spray liquid containing the coating agent in a spray liquid preparation solvent.

14. The oral preparation according to claim 13, wherein the spray liquid further comprises a lubricant and/or a low-viscosity binder.

15. The oral preparation according to claim 13, wherein the coating agent is an acrylic polymer-based coating agent.

16. The oral preparation according to claim 13, wherein the coating agent is Eudragit NE.

17. The oral preparation according to claim 9, wherein the disintegrator has a carboxymethyl group and the disintegrator is carmellose, carmellose sodium, carmellose calcium, croscarmellose sodium, sodium carboxymethyl starch, or carboxy methyl ethyl cellulose.

18. A method for producing an oral preparation which comprises:
obtaining granules through granulation of a mixture containing a medicinal substance having an unpleasant taste and a first additive, and a binder in a granulation liquid preparation solvent;
coating the granules with a coating agent to obtain coated granules; and
tableting the coated granules with a second additive and a disintegrator.

19. The method according to claim 18, wherein the medicinal substance having an unpleasant taste contains one medicinal substance selected from donepezil hydrochloride, zolpidem tartrate, risperidone, or amlodipine besilate.

20. The method according to claim 19, wherein the medicinal substance having an unpleasant taste is donepezil hydrochloride.

21. The method according to claim 18, wherein the first additive is an excipient.

22. The method according to claim 18, wherein the coated granules are spray-coated granules with a spray liquid containing the coating agent in a spray liquid preparation solvent.

23. The method according to claim 22, wherein the spray liquid further comprises a lubricant and/or a low-viscosity binder.

24. The method according to claim 22, wherein the coating agent is an acrylic polymer-based coating agent.

25. The method according to claim 22, wherein the coating agent is Eudragit NE.

26. The method according to claim 18, wherein the disintegrator has a carboxymethyl group and the disintegrator is carmellose, carmellose sodium, carmellose calcium, croscarmellose sodium, sodium carboxymethyl starch, or carboxy methyl ethyl cellulose.
